(19) United States
(12) Patent Application Publication
(10) Pub. No.: US 2015/0065802 A1
(43) Pub. Date: Mar. 5, 2015

(54) LIGHT SOURCE APPARATUS AND ENDOSCOPE SYSTEM

(71) Applicant: FUJIFILM Corporation, Tokyo (JP)

(72) Inventors: Satoshi OZAWA, Ashigarakami-gun (JP); Eiji OHASHI, Ashigarakami-gun (JP); Yoshinori MORIMOTO, Ashigarakami-gun (JP)

(21) Appl. No.: 14/319,488

(22) Filed: Jun. 30, 2014

(30) Foreign Application Priority Data
Aug. 27, 2013 (JP) 2013-175616

(51) Int. Cl. A61B 1/06

(52) U.S. Cl. CPC A61B 1/0661; A61B 1/0638
USPC 600/180; 600/178

(57) ABSTRACT

A light source apparatus outputs light for an endoscope apparatus. A blue LED emits blue light with a peak wavelength equal to or longer than 450 nm. A wavelength cut-off filter is disposed downstream of the blue LED, for cutting off a component in the blue light having a wavelength equal to or longer than the peak wavelength. Preferably, the wavelength cut-off filter cuts off a component of a wavelength equal to or longer than a reference wavelength in a wavelength range equal to or longer than the peak wavelength. A mode changer changes over an enhancement mode and a normal mode, the enhancement mode is set for enhancing and imaging a surface blood vessel of the object by use of the wavelength cut-off filter, the normal mode is set for imaging the object by disabling a function of the wavelength cut-off filter.

### CROSS-REFERENCE TO RELATED APPLICATION

This application claims priority under 35 USC 119 from Japanese Patent Application No. 2013-175616, filed 27 Aug. 2013, the disclosure of which is incorporated by reference herein.

### BACKGROUND OF THE INVENTION

1. Field of the Invention

The present invention relates to a light source apparatus and endoscope system. More particularly, the present invention relates to a light source apparatus and endoscope system, in which surface blood vessels in a body cavity can be imaged with high contrast and also a light amount of light for illumination can be maintained largely.

2. Description Related to the Prior Art

An endoscope system is well-known in the medical field for endoscopic imaging of a patient's body. The endoscope system includes an endoscope apparatus, a light source apparatus, and a processing apparatus. The light source apparatus supplies the endoscope apparatus with illumination light. The processing apparatus processes an image signal output by the endoscope apparatus. The endoscope apparatus includes an elongated tube for entry in a body cavity. A tip device of the elongated tube includes lighting window areas and a viewing window area. The lighting window areas apply the illumination light to an object of interest. The viewing window area receives object light for imaging. A light guide device is incorporated in the endoscope apparatus, and includes a fiber bundle in which plural optical fibers are bundled. The light guide device guides the illumination light from the light source apparatus to the light windows. An image sensor is disposed behind the viewing window area, such as a CCD image sensor. The object of interest illuminated with the illumination light is imaged by the image sensor. The processing apparatus produces an endoscopic image according to an image signal output by the image sensor. A physician or user can view the object of interest in the body as the image is displayed on a monitor display panel.

It is also widely known in the endoscopic imaging to use special light (narrow band light) with a limited wavelength range in contrast with the endoscopic imaging in which white light is used to image an entirety of a surface of body tissue. Various examples of the imaging with the special light are known. U.S. Pat. Nos. 8,337,400 and 8,506,478 (corresponding to JP-A 2009-297290) disclose image enhancement in which surface blood vessels on the surface of mucosa are enhanced and expressed by utilizing optical characteristic of the body tissue with a difference in penetration depth of light according to a wavelength of the light. A state of the blood vessels is different between normal body tissue and abnormal body tissue of a cancer, malignant tumor and the like. The image enhancement of blood vessels is very effective in discovery of a cancer at an early stage.

U.S. Pat. Nos. 8,337,400 and 8,506,478 disclose blue and green semiconductor light sources. The blue source includes a blue LED (light emitting diode) for emitting blue light in a narrow band with a wavelength of 450-480 nm at a half width and a peak wavelength of 465-470 nm. The green source includes a green LED (light emitting diode) for emitting green light in a narrow band with a wavelength of 520-560 nm at a half width and a peak wavelength of 530-535 nm. The LEDs are turned on to apply blue and green light to the object of interest simultaneously. The image sensor detects reflected light from the object of interest to acquire an image after enhancement of the surface blood vessels.

In U.S. Pat. Nos. 8,337,400 and 8,506,478, there is a disclosure that a preferable width of the wavelength range of light emitted by the LEDs is equal to or more than 10 nm in view of a sufficient light amount. Also, it is disclosed that the wavelength range of light emitted by the LEDs should be predetermined suitably at a narrow width in order to extract information from a tissue layer of interest in a selective manner.

In FIG. 24, spectral distributions of light reflected by the surface blood vessels and mucosa are illustrated. A difference between the spectral distribution is relatively large in the wavelength range shorter than 450 nm, and relatively small in the wavelength range equal to or longer than 450 nm. According to the observation of this difference, a light component of the wavelength range equal to or longer than 450 nm (characteristic wavelength) should be preferably small for the purpose of viewing the object of interest of the surface blood vessels, because an image of a high contrast can be acquired with distinction between the surface blood vessels and the mucosa.

In general, a light source for the endoscopic imaging should emit light at a relatively large light amount for the purpose of illuminating a body cavity as a dark area.

Presently available examples of blue LEDs have the peak wavelength equal to or longer than 450 nm on a condition of emitting light at a relatively large light amount sufficient for the endoscopic imaging. The blue LED disclosed U.S. Pat. Nos. 8,337,400 and 8,506,478 illuminates at the peak wavelength of approximately 465-470 nm.

The light emitted by the blue LED of the large light amount contains a component of the wavelength range equal to or longer than 450 nm which may lower the contrast of the surface blood vessels in the image. Cutting off of the component lowering the contrast will result in a sufficient contrast of the surface blood vessels. However, a component of the light at the peak wavelength is also cut off, so that the light amount may be insufficient for the endoscopic imaging due to a considerable loss in the light amount. There is no known technique for maintaining the contrast of the surface blood vessels at a high level and also ensuring the light amount sufficient for the endoscopic imaging.

U.S. Pat. Nos. 8,337,400 and 8,506,478 disclose the width of the wavelength range of light emitted by the LEDs is equal to or more than 10 nm for keeping the light amount, and predetermination of the wavelength range of light emitted by the LEDs at a narrow width for the purpose of extracting information from the tissue layer of interest selectively. However, the documents do not disclose an idea for maintaining the contrast of the surface blood vessels at a high level and also ensuring the light amount sufficient for the endoscopic imaging.

### SUMMARY OF THE INVENTION

In view of the foregoing problems, an object of the present invention is to provide a light source apparatus and endoscope system, in which surface blood vessels in a body cavity can be imaged with high contrast and also a light amount of light for illumination can be maintained largely.

In order to achieve the above and other objects and advantages of this invention, a light source apparatus for outputting illumination light for an endoscope apparatus is provided, and includes a blue semiconductor light source for emitting blue light with a peak wavelength equal to or longer than 450 nm. A wavelength cut-off filter is disposed downstream of the blue semiconductor light source, for cutting off a component in the blue light having a wavelength equal to or longer than the peak wavelength.

Preferably, the wavelength cut-off filter cuts off a component defined at a wavelength equal to or longer than the peak wavelength.

Preferably, the wavelength cut-off filter cuts off a component with a wavelength on a long wavelength side of a wavelength range of a half width of the blue light.

Preferably, furthermore, a mode changer changes over an enhancement mode and a normal mode, the enhancement mode being set for enhancing and imaging a surface blood vessel of the object by use of the wavelength cut-off filter, the normal mode being set for imaging the object by disabling a function of the wavelength cut-off filter.

Preferably, the mode changer includes a moving mechanism for moving the wavelength cut-off filter between an operative position inside a light path from the blue semiconductor light source and an inoperative position outside the light path. A controller drives the moving mechanism according to selection of the enhancement mode and the normal mode, sets the wavelength cut-off filter in the operative position in case of selecting the enhancement mode, and sets the wavelength cut-off filter in the inoperative position in case of selecting the normal mode.

Preferably, furthermore, a green semiconductor light source emits green light of a wavelength range of green. A red semiconductor light source emits red light of a wavelength range of red. An optical coupling device combines the blue, green and red light from the wavelength cut-off filter, the green semiconductor light source and the red semiconductor light source with one another, to output the illumination light.

Preferably, furthermore, a violet semiconductor light source emits violet light of a wavelength range of violet, to enhance a surface blood vessel of the object at least partially.

Preferably, the optical coupling device includes a first dichroic mirror for combining light of a first color with light of a second color, the first and second colors being selected from the blue, green and red. A second dichroic mirror combines combined light from the first dichroic mirror with a third color selected from the blue, green and red, to output the illumination light.

Preferably, the blue semiconductor light source has a blue light-emitting diode.

Preferably, the endoscope apparatus includes a light guide device for transmitting the illumination light in a distal direction, and a lighting window area for applying the illumination light from the light guide device to an object in a body cavity.

Preferably, the wavelength cut-off filter cuts off a component of a wavelength equal to or longer than a reference wavelength in a wavelength range equal to or longer than the peak wavelength.

Preferably, the reference wavelength is equal to the peak wavelength.

Preferably, the reference wavelength is equal to or shorter than a wavelength that is on a long wavelength side in a wavelength range of a half width of the blue light.

Also, an endoscope system having an endoscope apparatus and a light source apparatus for outputting illumination light for the endoscope apparatus is provided. The endoscope apparatus includes a light guide device for transmitting the illumination light in a distal direction. A lighting window area applies the illumination light from the light guide device to an object in a body cavity. The light source apparatus includes a blue semiconductor light source for emitting blue light with a peak wavelength equal to or longer than 450 nm. A wavelength cut-off filter is disposed downstream of the blue semiconductor light source, for cutting off a component in the blue light having a wavelength equal to or longer than the peak wavelength.

Accordingly, surface blood vessels in a body cavity can be imaged with high contrast and also a light amount of light for illumination can be maintained largely, because a wavelength cut-off filter cuts off a light component which may lower than contrast of an image.

### BRIEF DESCRIPTION OF THE DRAWINGS

The above objects and advantages of the present invention will become more apparent from the following detailed description when read in connection with the accompanying drawings, in which:

FIG. 1 is an explanatory view illustrating an endoscope system;

FIG. 2 is a front elevation illustrating a tip device of an endoscope apparatus;

FIG. 3 is a block diagram schematically illustrating circuit elements in the endoscope system;

FIG. 4 is a cross section illustrating a blue semiconductor light source;

FIG. 5 is a graph illustrating spectral distribution of blue light;

FIG. 6 is a graph illustrating spectral distribution of green light;

FIG. 7 is a graph illustrating spectral distribution of red light;

FIG. 8 is a graph illustrating a transmission characteristic of a wavelength cut-off filter;

FIG. 9 is a graph illustrating spectral distribution of filtered blue light;

FIG. 10 is a graph illustrating spectral distribution of the illumination light having filtered blue light and green and red light;

FIG. 11 is a graph illustrating spectral distribution of micro color filters of an image sensor;

FIG. 12 is a timing chart illustrating emission of light and imaging of the image sensor;

FIG. 13 is an explanatory view in elevation illustrating arrangement of semiconductor light sources and an optical coupling device;

FIG. 14 is a graph illustrating a transmission characteristic of a dichroic filter in a first dichroic mirror;

FIG. 15 is a graph illustrating a transmission characteristic of a dichroic filter in a second dichroic mirror;

FIG. 16 is an explanatory view in elevation illustrating another preferred embodiment with a mode changer;

FIG. 17 is a graph illustrating spectral distribution of the illumination light having the blue, green and red light;

FIG. 18 is an explanatory view in elevation illustrating still another preferred embodiment having a violet semiconductor light source;

FIG. 19 is a graph illustrating spectral distribution of violet light;

FIG. 20 is a graph illustrating spectral distribution of the illumination light having filtered blue light and green, red and violet light;

FIG. 21 is a graph illustrating a transmission characteristic of a dichroic filter in a dichroic mirror;

FIG. 22 is a graph illustrating a scattering coefficient of body tissue;

FIG. 23 is a timing chart illustrating emission of light and imaging of the image sensor in image enhancement with the violet light;

FIG. 24 is a graph illustrating reflection spectrum of surface blood vessels and mucosa.

### DETAILED DESCRIPTION OF THE PREFERRED EMBODIMENT(S) OF THE PRESENT INVENTION

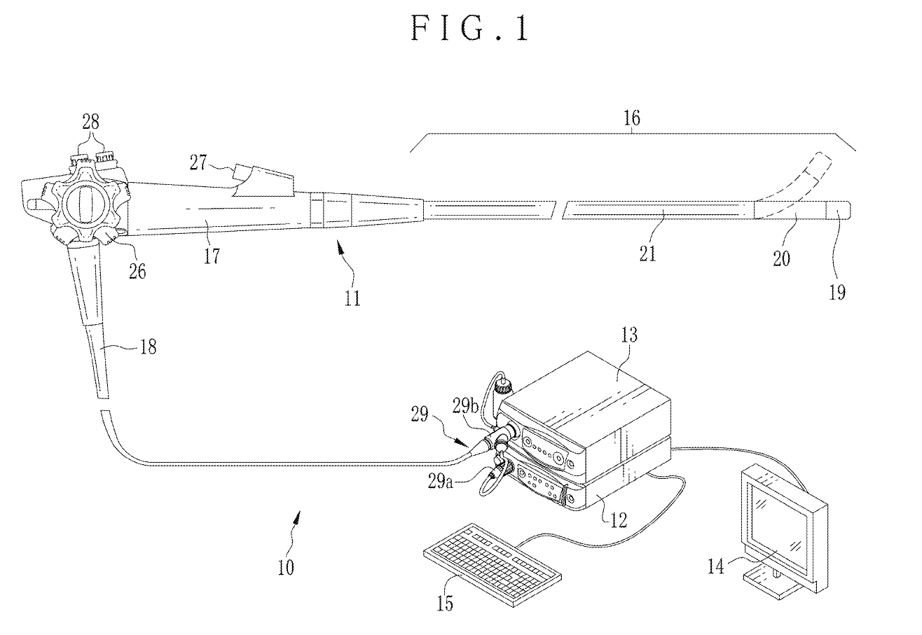

In FIG. 1, an endoscope system 10 includes an endoscope apparatus 11, a processing apparatus 12, a light source apparatus 13, and a monitor display panel 14. The endoscope apparatus 11 as a medical apparatus images an object of interest in a body cavity, for example, gastrointestinal tract. The processing apparatus 12 produces an endoscopic image of the object of interest according to an image signal obtained by imaging. The light source apparatus 13 supplies the endoscope apparatus 11 with illumination light for lighting the body cavity. The monitor display panel 14 displays the endoscopic image. A user input interface 15 is connected to the processing apparatus 12, for example, keyboard, mouse and the like.

The endoscope apparatus 11 includes an elongated tube 16, a grip handle 17 and a universal cable 18. The elongated tube 16 is entered in the body cavity of a patient's body. The grip handle 17 is disposed at a proximal end of the elongated tube 16. The universal cable 18 connects the endoscope apparatus 11 to the processing apparatus 12 and the light source apparatus 13.

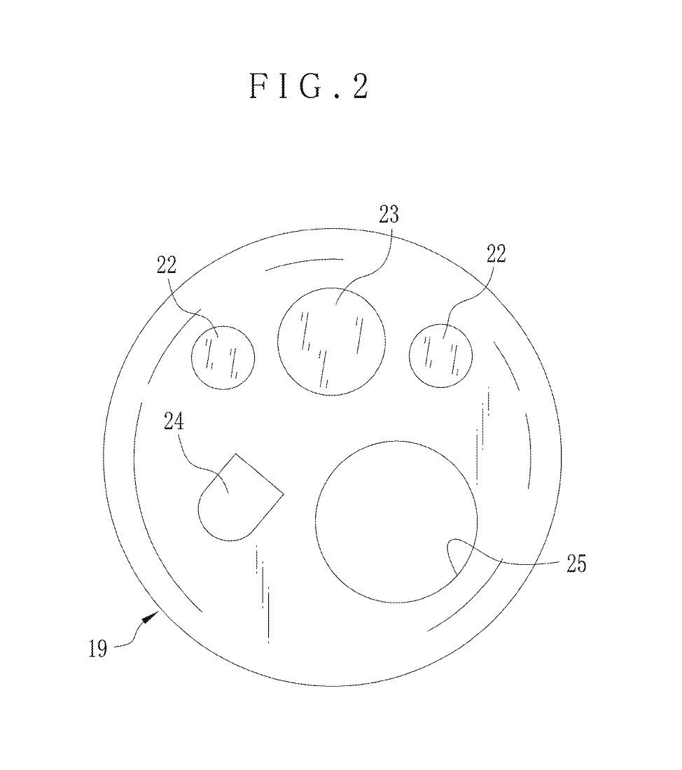

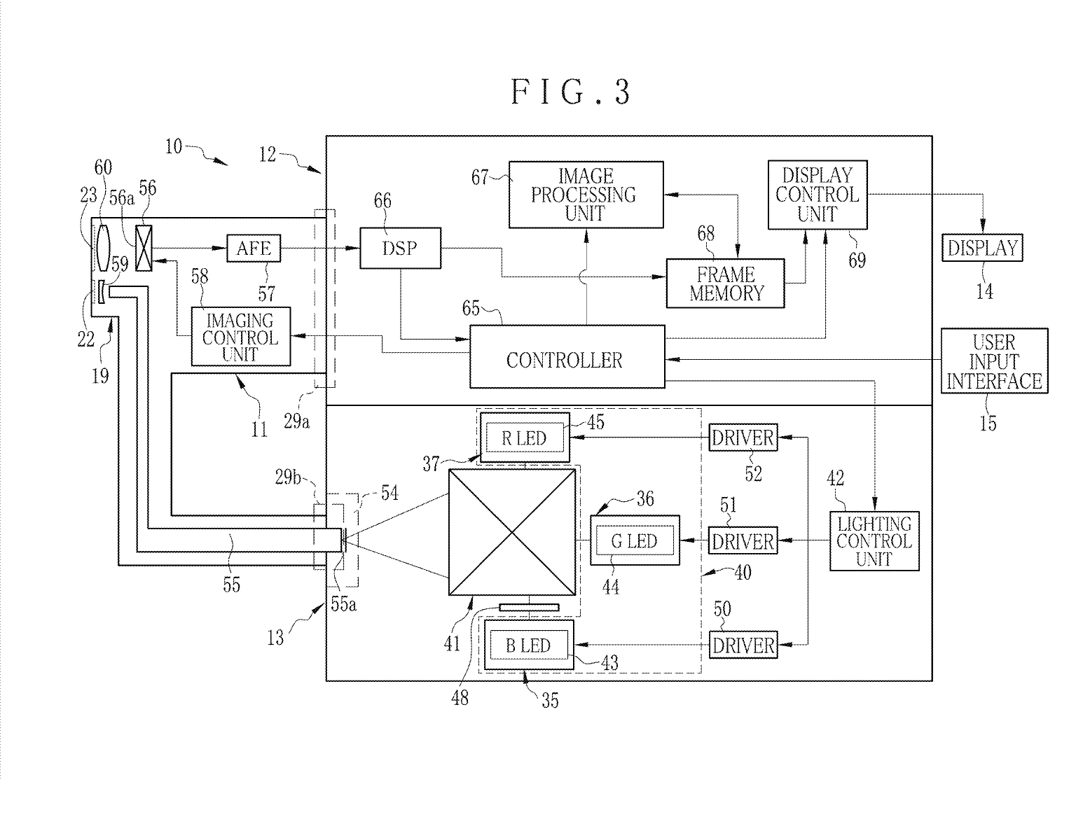

The elongated tube 16 includes a tip device 19, a steering device 20 and a flexible device 21 arranged in a proximal direction. In FIG. 2, various elements are disposed on an end surface of the tip device 19, including lighting window areas 22, a viewing window area 23, a nozzle spout 24 and a distal instrument opening 25. The lighting window areas 22 apply illumination light to an object of interest. The viewing window area 23 receives image light from the object of interest. The nozzle spout 24 supplies air or water to clean up the viewing window area 23. The distal instrument opening 25 is used to protrude a medical instrument such as a forceps, electrocautery device and the like for treatment of various types. An image sensor 56 and a lens system 60 or objective are disposed behind the viewing window area 23. See FIG. 3

The steering device 20 is constituted by a plurality of link elements connected serially. Steering wheels 26 are disposed on the grip handle 17, and rotated to bend the steering device 20 up and down and to the right and left. The tip device 19 is directed in a desired direction by steering of the steering device 20. The flexible device 21 is flexible for entry in a body cavity of a tortuous shape, for example, esophagus or intestines in a gastrointestinal tract. A communication cable, a light guide device 55 and the like are entered through the elongated tube 16 as illustrated in FIG. 3. The communication cable transmits a drive signal for driving the image sensor 56, and an image signal output by the image sensor 56. The light guide device 55 transmits light from the light source apparatus 13 to the lighting window areas 22.

The grip handle 17 includes a proximal instrument opening 27, fluid supply buttons 28 and a release button (not shown) in addition to the steering wheels 26. The proximal instrument opening 27 receives entry of a medical instrument for treatment. The fluid supply buttons 28 are depressed for supplying air or water through the nozzle spout 24. The release button is depressible for forming a still image.

Various elements are contained in the tube of the universal cable 18, including a communication cable extending from the elongated tube 16, the light guide device 55 and the like. A composite plug 29 or composite connector is disposed at a proximal end of the universal cable 18 directed to the processing apparatus 12 and the light source apparatus 13. The composite plug 29 includes a communication connector 29a and a light source connector 29b. The communication connector 29a is coupled to the processing apparatus 12 removably. The light source connector 29b is coupled to the light source apparatus 13 removably. A proximal end of the communication cable is disposed in the communication connector 29a. An entrance end 55a of the light guide device 55 of FIG. 3 is disposed in the light source connector 29b.

In FIG. 3, the light source apparatus 13 includes an illuminator 40 or light source unit, an optical coupling device 41 or light coupling device, and a lighting control unit 42. The illuminator 40 includes a blue semiconductor light source 35, a green semiconductor light source 36 and a red semiconductor light source 37. The optical coupling device 41 combines light paths downstream of the semiconductor light sources 35-37. The lighting control unit 42 controls the semiconductor light sources 35-37.

The blue semiconductor light source 35 includes a blue LED 43 or blue light emitting device for emitting light of a wavelength range of blue. The green semiconductor light source 36 includes a green LED 44 or green light emitting device for emitting light of a wavelength range of green. The red semiconductor light source 37 includes a red LED 45 or red light emitting device for emitting light of a wavelength range of red. Each of the color LEDs 43-45 has a p-type semiconductor and an n-type semiconductor attached together as is well-known in the art. Upon application of voltage, recombination of a positive hole and an electron occurs across the band gap at the p-n junction, for a current to flow. Light is emitted by generation of energy according to the band gap. A light amount emitted by the color LEDs 43-45 is increased by an increase in the voltage.

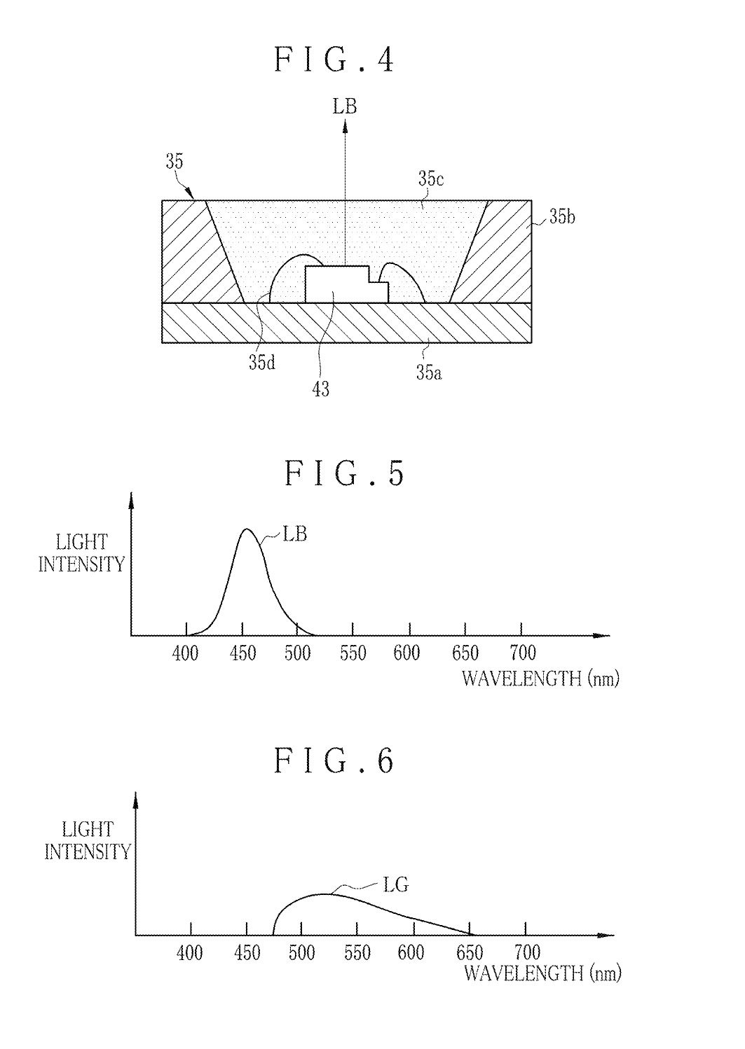

In FIG. 4, the blue semiconductor light source 35 includes a semiconductor substrate 35a or board, a cavity mold 35b and a resin encapsulant 35c. On the semiconductor substrate 35a is mounted the blue LED 43. The cavity mold 35b is formed on the semiconductor substrate 35a and has a cavity for containing the blue LED 43. The resin encapsulant 35c is disposed in the cavity. An inner surface of the cavity reflects light as a reflector cup. A diffusing material is mixed in the resin encapsulant 35c for diffusing light. Semiconductor wiring 35d or wires connect the blue LED 43 to the semiconductor substrate 35a. Amounting type of the blue semiconductor light source 35 is referred to technically as a surface mounting type. Note that the semiconductor light sources 35-37 are structurally the same. The blue semiconductor light source 35 is described among those, the green and red semiconductor light sources 36 and 37 being not described further.

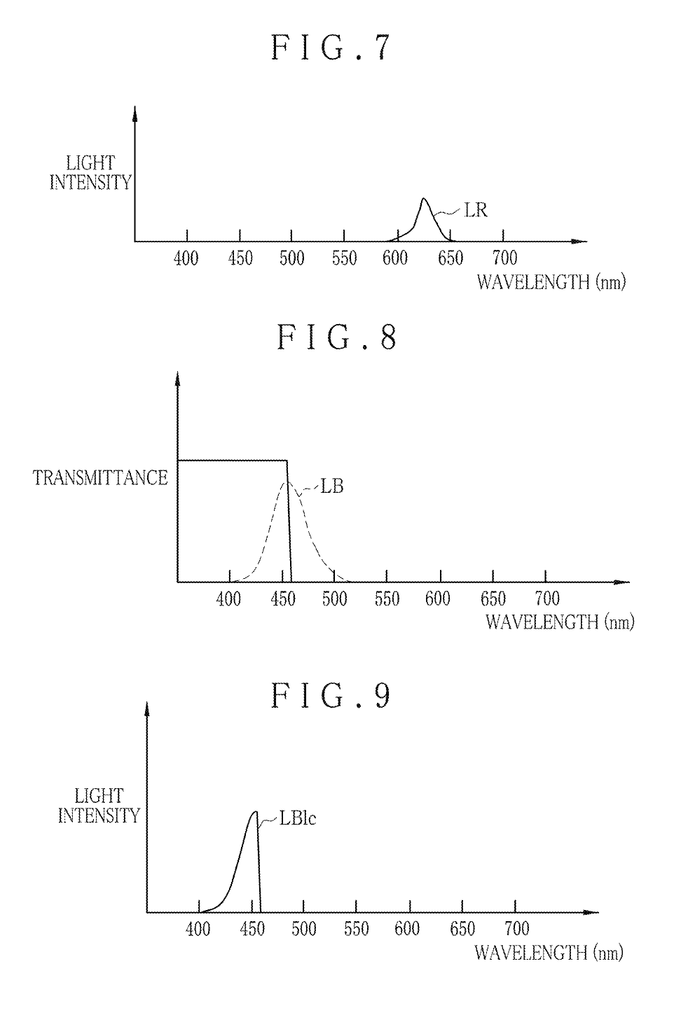

In FIG. 5, the blue semiconductor light source 35 emits blue light LB of a wavelength of 440-470 nm as a blue range, with a center wavelength of 455±10 nm and a peak wavelength of 455 nm. In FIG. 6, the green semiconductor light source 36 emits green light LG of a wavelength of 500-600 nm as a green range, with a center wavelength of 520±10 nm and a peak wavelength of 520 nm. In FIG. 7, the red semiconductor light source 37 emits red light LR of a wavelength of 615-635 nm as a red range, with a center wavelength of 620±10 nm and a peak wavelength of 625 nm. Note that the center wavelength is a wavelength at the center of the width of an emission spectrum of the light. The peak wavelength is a wavelength at a vertex of the emission spectrum of the light.

In FIG. 3, a wavelength cut-off filter 48 (short wavelength pass filter) or long cut filter (LCF) is disposed in front of the blue semiconductor light source 35. The wavelength cut-off filter 48 cuts off a long wavelength component of a wavelength longer than 455 nm (reference wavelength) as peak wavelength from the blue light LB from the blue semiconductor light source 35. In FIG. 8, the wavelength cut-off filter 48 reflects light in a wavelength range longer than 455 nm in the green and red colors, and transmits light in a wavelength range shorter than 455 nm in the blue color.

The wavelength cut-off filter 48 obtains the filtered blue light LB1c of FIG. 9 from the blue light LB. The filtered blue light LB1c is light of a relatively large light amount, and obtained by cutting off most of a first component of the blue light LB and includes a second component, the first component having a wavelength range equal to or longer than 450 nm (characteristic wavelength) as an obstacle to raising a contrast of surface blood vessels (See FIG. 24), the second component having a peak wavelength of 455 nm in the blue light LB. The filtered blue light LB1c becomes incident upon the optical coupling device 41.

Drivers 50, 51 and 52 are connected to respectively the color LEDs 43-45, and are caused by the lighting control unit 42 to control the color LEDs 43-45 for turning on and off and adjusting light amounts. For the adjustment, power for the color LEDs 43-45 is adjusted according to a control signal from the processing apparatus 12.

The drivers 50-52 are controlled by the lighting control unit 42, and continuously supply the color LEDs 43-45 with drive currents to turn on those. According to an exposure control signal received from the processing apparatus 12, power supplied to the color LEDs 43-45 is changed by changing the drive current, to adjust light amounts of the blue light LB, green light LG and red light LR. Note that the drive current may not be supplied continuously but in a pulsed manner. It is possible to perform PAM control (pulse amplitude modulation) or PWM control (pulse width modulation). In the PAM control, an amplitude of the drive current pulse is changed. In the PWM control, a duty factor of the drive current pulse is changed.

The optical coupling device 41 combines light paths of the filtered blue light LB1c, green light LG and red light LR and forms one light path as an exit end. There is a receptacle connector 54 for coupling of the light source connector 29b. The exit end of the optical coupling device 41 is disposed near to the receptacle connector 54. The optical coupling device 41 receives incident light from the semiconductor light sources 35-37 and exits the light toward the entrance end 55a of the light guide device 55 of the endoscope apparatus 11. Glass covers (not shown) are disposed with respectively the light source connector 29b and the receptacle connector 54.

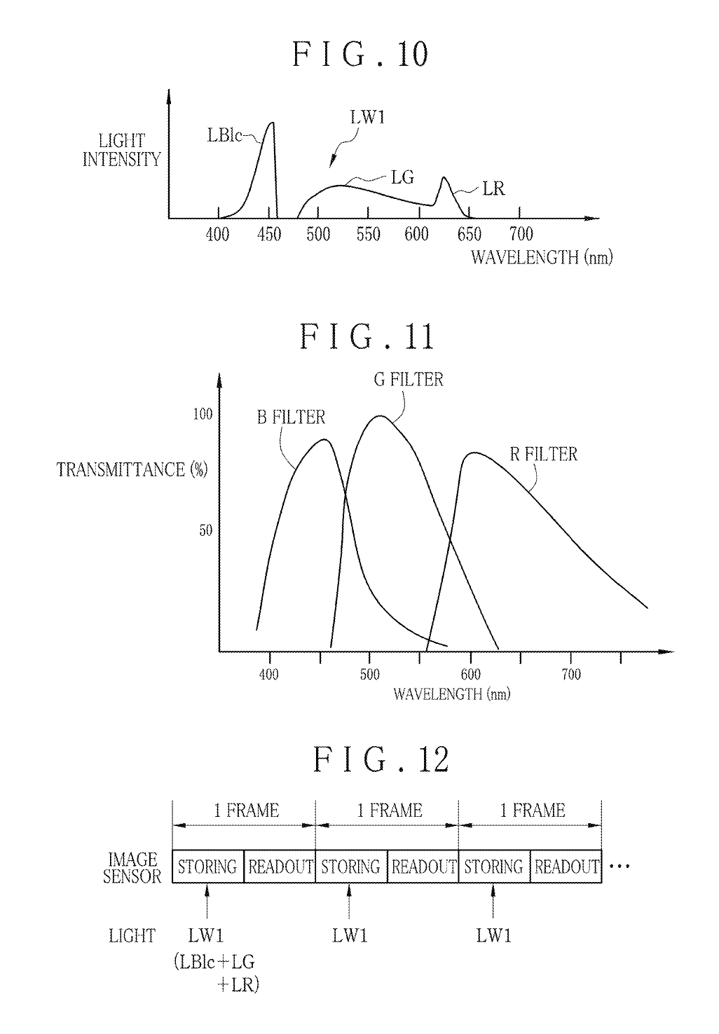

The spectral distribution of combined light (mixed light) containing the filtered blue light LB1c, the green light LG and the red light LR from the optical coupling device 41 is illustrated in FIG. 10. The combined light is used as the illumination light LW1. The feature in FIG. 10 is only one example. The spectral distribution of the illumination light LW1 as a target can be changed according to color balance of a desired endoscopic image. For example, a ratio between the light amounts of the filtered blue light LB1c, the green light LG and the red light LR is changed by changing currents to drive the color LEDs 43-45, to emit the illumination light LW1 with the target spectral distribution.

The lighting control unit 42 maintains spectral distribution of target and performs exposure control of the illumination light. Should a ratio change between light amounts of colors in the illumination light, the spectral distribution of the illumination light is changed to change color balance of an endoscopic image. In view of this, the lighting control unit 42 changes drive currents for the color LEDs 43-45 discretely by use of the drivers 50-52 to regulate the ratio between the light amounts of colors, so as to increase or decrease the light amounts.

The endoscope apparatus 11 includes an analog processing unit 57 or analog front end (AFE) and an imaging control unit 58 in addition to the light guide device 55 and the image sensor 56. The light guide device 55 is a fiber bundle constituted by bundling plural optical fibers. Upon coupling the light source connector 29b to the light source apparatus 13, the entrance end 55a of the light guide device 55 in the light source connector 29b is aligned with an exit end of the optical coupling device 41. A distal exit end of the light guide device 55 inside the tip device 19 has two branches for transmitting light to the lighting window areas 22.

An illumination lens 59 is disposed behind the lighting window areas 22. Illumination light from the light source apparatus 13 is guided by the light guide device 55 to the illumination lens 59, and applied through the lighting window areas 22 to an object of interest. The illumination lens 59 is a concave lens and enlarges a divergence angle of light from the light guide device 55. The illumination light can be applied to a wide area in a body cavity with the object of interest.

The lens system 60 and the image sensor 56 are disposed behind the viewing window area 23. Object light from a body cavity enters the lens system 60 through the viewing window area 23, and is focused on an imaging surface 56a of the image sensor 56 by the lens system 60.

Examples of the image sensor 56 are a CCD image sensor and CMOS image sensor. A plurality of photoconductive elements or photoconductors are arranged as pixels on the imaging surface 56a, for example, photo diodes. The image sensor 56 photoelectrically converts light received by the imaging surface 56a, and stores signal charge according to light amounts of light received by the pixels. The signal charge is converted by an amplifier into a voltage signal, which is read out. The voltage signal is transmitted by the image sensor 56 to the analog processing unit 57 as an image signal.

The analog processing unit 57 is constituted by a correlated double sampler, auto gain controller and A/D converter (all not shown). The correlated double sampler processes the image signal of an analog form from the image sensor 56 in the correlated double sampling, and removes electric noise due to reset of the signal charge. The auto gain controller amplifies the image signal after removal of the noise in the correlated double sampler. The A/D converter converts the amplified image signal from the auto gain controller into a digital image signal having a gradation value according to a predetermined bit number, and sends the digital image signal to the processing apparatus 12.

A controller 65 in the processing apparatus 12 is connected with the imaging control unit 58, which supplies the image sensor 56 with a drive signal according to a clock signal from the controller 65 as a reference. The image sensor 56 generates an image signal at a predetermined frame rate according to the drive signal from the imaging control unit 58, and sends the image signal to the analog processing unit 57.

The image sensor 56 is a color imaging unit. Micro color filters of blue, green and red colors are disposed on the imaging surface 56a with spectral characteristics of FIG. 11, and associated with pixels. An example of arrangement of the micro color filters is Bayer arrangement.

The B pixels with the B filters are sensitive to light of a wavelength of approximately 380-560 nm. The G pixels with the G filters are sensitive to light of a wavelength of approximately 450-630 nm. The R pixels with the R filters are sensitive to light of a wavelength of approximately 580-800 nm. The illumination light LW1 is constituted by the filtered blue light LB1c, the green light LG and the red light LR. Reflected light corresponding to the filtered blue light LB1c is mainly received by the B pixels. Reflected light corresponding to the green light LG is mainly received by the G pixels. Reflected light corresponding to the red light LR is mainly received by the R pixels.

During the period of acquiring one frame in FIG. 12, the image sensor 56 carries out storing and readout. In the storing, the red LED 45 stores signal charge in pixels. In the readout, the signal charge is read out from the pixels. According to the time period of storing in the image sensor 56, the semiconductor light sources 35-37 are turned on to apply illumination light LW1 as a mixture of the filtered blue light LB1c, the green light LG and the red light LR to the object of interest. Reflected light from the object of interest becomes incident upon the image sensor 56. In the image sensor 56, the micro color filters carry out color separation of the reflected light of the illumination light LW1. B pixels receive the reflected light according to the filtered blue light LB1c. G pixels receive the reflected light according to the green light LG. R pixels receive the reflected light according to the red light LR. In synchronism with the readout, the image sensor 56 sequentially outputs image signals B, G and R according to the frame rate for one frame in which pixel values of B, G and R pixels are combined.

In FIG. 3, the processing apparatus 12 includes a digital signal processor 66 (DSP), an image processing unit 67, a frame memory 68 and a display control unit 69 together with the controller 65. The controller 65 has a CPU with a ROM and a RAM. The ROM stores control programs and control data. The RAM is a working memory for loading of the control program. The CPU runs the control program to control various elements in the processing apparatus 12.

The digital signal processor 66 obtains an image signal output by the image sensor 56. The digital signal processor 66 separates the image signal of mixture according to the pixels of B, G and R into image signals of B, G and R, and processes those image signals in pixel interpolation. Also, the digital signal processor 66 carries out signal processing of those image signals for gamma correction, white balancing and the like.

The digital signal processor 66 determines an exposure amount according to the image signals B, G and R. Should the exposure amount of the entirety of the image be too low (underexposure), the digital signal processor 66 outputs a control signal to the controller 65 to raise the exposure amount of the illumination light. Should the exposure amount of the entirety of the image be too high (overexposure), the digital signal processor 66 outputs a control signal to the controller 65 to lower the exposure amount of the illumination light. The controller 65 sends a control signal to the lighting control unit 42 of the light source apparatus 13.

The frame memory 68 stores image data output by the digital signal processor 66, processed image data processed by the image processing unit 67, and the like. The display control unit 69 reads out the processed image data from the frame memory 68, converts the same into a composite signal, component signal or other video signal, and outputs the signal to the monitor display panel 14.

The image processing unit 67 produces an endoscopic image according to the image signals B, G and R after color separation by the digital signal processor 66. The endoscopic image is output to the monitor display panel 14. At each time that the image signals B, G and R in the frame memory 68 are updated, the image processing unit 67 updates the endoscopic image. The image signal B is formed by receiving a reflected light component according to the filtered blue light LB1c in the illumination light LW1. Surface blood vessels are expressed at a high contrast with the image signal B. It is medically known that there is a characteristic pattern of the particular surface blood vessels in body tissue of a cancer, malignant tumor or other lesions, because higher vessel density of the particular surface blood vessels is found than normal body tissue. It is desirable to express the surface blood vessels distinctly for the diagnosis of a benign or malignant tumor.

It is also possible to extract an area of the surface blood vessels within the endoscopic image according to the image signal B, and process the area of the surface blood vessels in the edge enhancement. The image signal B after the edge enhancement is combined with a full color image according to the image signals B, G and R.

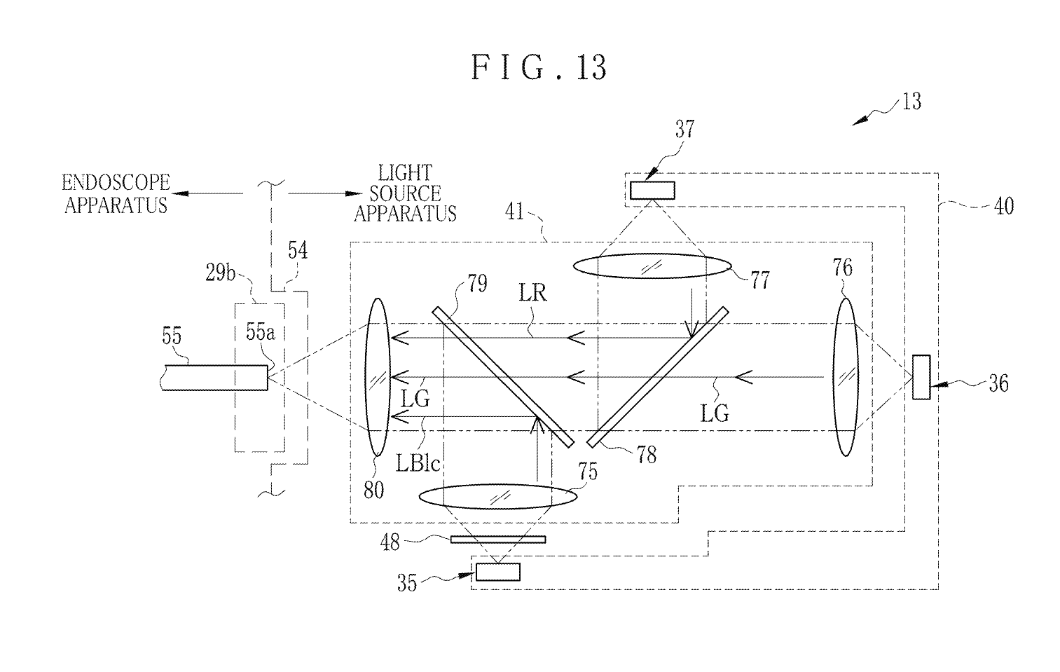

In FIG. 13, the optical coupling device 41 includes collimator lenses 75, 76 and 77, first and second dichroic mirrors 78 and 79 and a condenser lens 80. The collimator lenses 75-77 collimate light from respectively the semiconductor light sources 35-37. The condenser lens 80 condenses light output by the optical coupling device 41 in a direction toward the entrance end 55a of the light guide device 55. Each of the first and second dichroic mirrors 78 and 79 includes a transparent plate of glass and a dichroic filter formed on the plate with a characteristic of predetermined transmittance.

The green semiconductor light source 36 is axially aligned with the light guide device 55. The green and red semiconductor light sources 36 and 37 are so disposed that their optical axes are perpendicular to one another. The first dichroic mirror 78 is positioned at an intersection of those optical axes. Also, the blue and green semiconductor light sources 35 and 36 are so disposed that their optical axes are perpendicular to one another. The second dichroic mirror 79 is positioned at an intersection of those optical axes. The first dichroic mirror 78 is inclined at 45 degrees with reference to the optical axes of the green and red semiconductor light sources 36 and 37. The second dichroic mirror 79 is inclined at 45 degrees with reference to the optical axes of the blue and green semiconductor light sources 35 and 36.

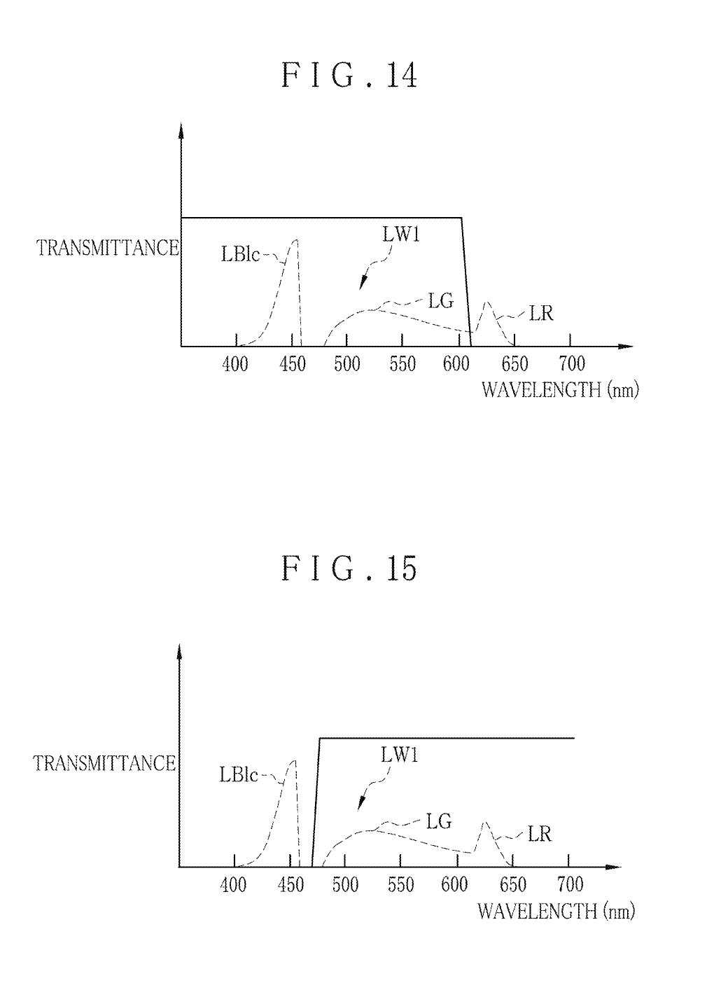

In FIG. 14, the dichroic filter of the first dichroic mirror 78 characteristically reflects light of a wavelength longer than approximately 610 nm in a red color and transmits light of a wavelength shorter than approximately 610 nm in blue and green colors. The first dichroic mirror 78 transmits the green light LG from the green semiconductor light source 36 through the collimator lens 76, and reflects the red light LR from the red semiconductor light source 37 and the collimator lens 77. The first dichroic mirror 78 combines light paths of the green and red light LG and LR with one another.

In FIG. 15, the dichroic filter of the second dichroic mirror 79 characteristically reflects light of a wavelength shorter than approximately 470 nm in a blue color and transmits light of a wavelength equal to or longer than approximately 470 nm in green and red colors. The second dichroic mirror 79 transmits the green light LG passed through the first dichroic mirror 78 and the red light LR reflected by the first dichroic mirror 78. Also, the second dichroic mirror 79 reflects the filtered blue light LB1c from the wavelength cut-off filter 48 and the collimator lens 75. The second dichroic mirror 79 combines light paths of the filtered blue light LB1c, the green light LG and the red light LR with one another to produce the illumination light LW1 or available light.

The operation of the above construction is described now. For endoscopic diagnosis, the endoscope apparatus 11 is connected to the processing apparatus 12 and the light source apparatus 13. A power source of the processing apparatus 12 and the light source apparatus 13 is turned on to start up the endoscope system 10.

The elongated tube 16 of the endoscope apparatus 11 is entered in a gastrointestinal tract of a body to start imaging of an object of interest. The lighting control unit 42 sets values of drive currents for the color LEDs 43-45, and starts turning on the semiconductor light sources 35-37. Their light amounts are controlled while spectral distribution of emitted light as target is maintained.

In the semiconductor light sources 35-37, the color LEDs 43-45 emit the blue light LB, green light LG and red light LR. The blue light LB is transmitted through the wavelength cut-off filter 48 and converted into the filtered blue light LB1c. The filtered blue light LB1c, green light LG and red light LR become incident upon the collimator lenses 75-77 in the optical coupling device 41.

The blue light LB is in a wavelength range of 440-470 nm and has a peak wavelength of 455 nm. It is preferable to cut off a component in the blue light LB of a wavelength equal to or longer than 450 nm (characteristic wavelength) for the purpose of expressing surface blood vessels at a high contrast by enhancing a difference in contrast between those and mucosa as illustrated in FIG. 24. However, wavelength cut-off with reference to 450 nm may lower the light amount remarkably because a component at the peak wavelength of 455 nm is not transmitted. In view of this, a reference wavelength for wavelength cut-off with the wavelength cut-off filter 48 is set at 455 nm longer than 450 nm, so that a light amount desired for endoscopic imaging is ensured. Thus, it is possible to raise the contrast of the surface blood vessels and ensuring the light amount in a well-balanced manner.

The filtered blue light LB1c is reflected by the second dichroic mirror 79. The green light LG travels through the first and second dichroic mirrors 78 and 79. The red light LR is reflected by the first dichroic mirror 78 and travels through the second dichroic mirror 79. The first and second dichroic mirrors 78 and 79 combine light paths of the filtered blue light LB1c, the green light LG and the red light LR. The filtered blue light LB1c, the green light LG and the red light LR become incident upon the condenser lens 80. Thus, the illumination light LW1 is produced from the filtered blue light LB1c, the green light LG and the red light LR. The condenser lens 80 condenses the illumination light LW1 in a direction toward the entrance end 55a of the light guide device 55 of the endoscope apparatus 11 to supply the endoscope apparatus 11 with the illumination light LW1.

In the endoscope apparatus 11, the illumination light LW1 is directed to the lighting window areas 22 by the light guide device 55 and applied to an object of interest. Reflected light of the illumination light LW1 from the object of interest is entered in the viewing window area 23 and detected by the image sensor 56. The image sensor 56 outputs image signals B, G and R to the digital signal processor 66 of the processing apparatus 12. The digital signal processor 66 separates the image signals B, G and R in the color separation, and transmits those to the image processing unit 67. Imaging of the image sensor 56 is repeated at a predetermined frame rate. The image processing unit 67 produces an endoscopic image according to the image signals B, G and R. The display control unit 69 outputs the endoscopic image to the monitor display panel 14. The endoscopic image is updated according to the frame rate of the image sensor 56.

The digital signal processor 66 determines an exposure amount according to the image signals B, G and R, and sends a control signal to the lighting control unit 42 of the light source apparatus 13. The lighting control unit 42 determines drive currents for the semiconductor light sources 35-37 so as to keep a ratio between light amounts of the colors at a constant level according to the received control signal (so as to keep the spectral distribution unchanged as a target). Then the semiconductor light sources 35-37 are driven with the determined drive currents. It is possible in the semiconductor light sources 35-37 to keep the light amounts of the filtered blue light LB1c, green light LG and red light LR in the illumination light LW1 at the constant level suitable for imaging.

The filtered blue light LB1c contained in the illumination light LW1 or available light does not have a component lowering the contrast of surface blood vessels in the image. The filtered blue light LB1c has a large light amount enough for endoscopic imaging. Thus, a difference between the surface blood vessels and the mucosa can be discerned, so as to obtain an endoscopic image with sufficient brightness.

The position of the wavelength cut-off filter 48 is not limited to that between the blue semiconductor light source 35 and the collimator lens 75 in the first embodiment, but can be set on a light path of the blue light LB. For example, the wavelength cut-off filter 48 may be disposed between the collimator lens 75 and the second dichroic mirror 79. The reference wavelength of cutting off in the wavelength cut-off filter 48 is not limited to the peak wavelength of the first embodiment, but can be longer than the peak wavelength. Should the reference wavelength be too long, a contrast of surface blood vessels will be low in spite of an ensured light amount. It is preferable to determine the reference wavelength by considering consistency between the contrast of the surface blood vessels and the light amount. For example, the reference wavelength can be equal to or shorter than a wavelength on a long wavelength side of a range of a wavelength of a half width of the blue light LB, for example, equal to or shorter than approximately 470 nm in the first embodiment.

With this reference wavelength, the wavelength cut-off filter 48 is effective in cutting off a component with the wavelength on the long wavelength side of the wavelength range of the half width of the blue light LB.

Furthermore, a reference wavelength for wavelength cut-off can be predetermined between 450 nm (characteristic wavelength) and the peak wavelength.

Also, it is possible to prepare a plurality of wavelength cut-off filters (short wavelength pass filters) or long cut filters (LCF), which are different in the reference wavelength from one another, and to use those selectively by changeover in compliance with light intensity of the light or according to user preference.

### Second Preferred Embodiment

In the first embodiment, the wavelength cut-off filter 48 is stationary at the blue semiconductor light source 35. However, it is possible to change over the wavelength cut-off filter 48 in operative and inoperative positions for cutting off.

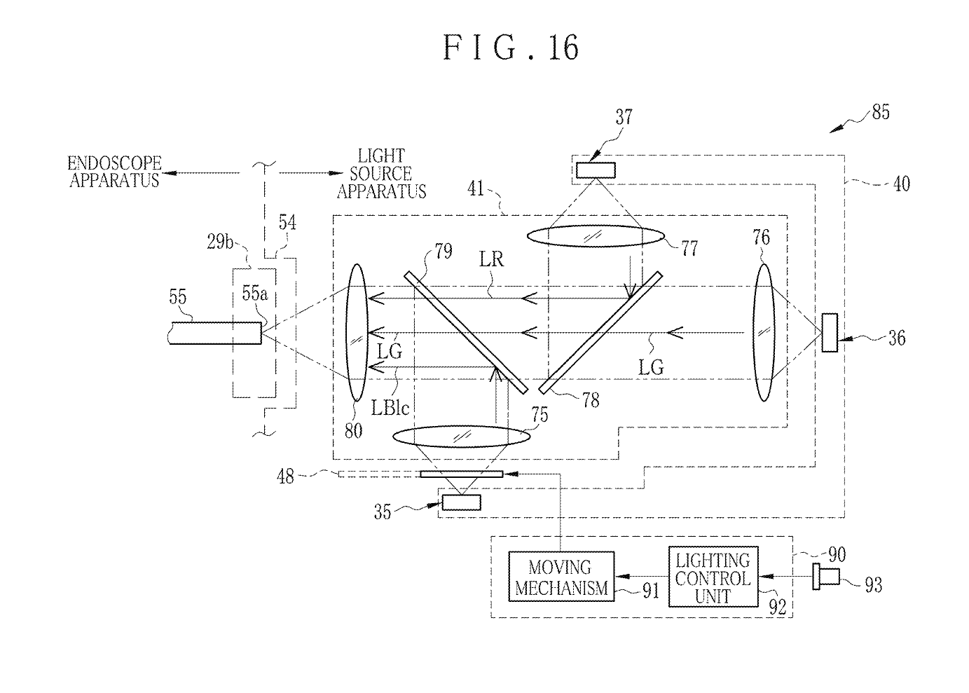

In FIG. 16, a light source apparatus 85 has a mode changer 90, which sets one of an enhancement mode and a normal mode selectively. In the enhancement mode, the wavelength cut-off of the wavelength cut-off filter 48 is enabled to enhance surface blood vessels. In the normal mode, the wavelength cut-off of the wavelength cut-off filter 48 is disabled to view the entirety of the object of interest normally. Except for the mode changer 90, the light source apparatus 13 of the first embodiment is repeated in the light source apparatus 85. Elements similar to those of the above embodiment are designated with identical reference numerals.

The mode changer 90 includes a moving mechanism 91 for the wavelength cut-off filter (short wavelength pass filter) or long cut filter (LCF), and a lighting control unit 92. A mode change button 93 is connected to the lighting control unit 92. The mode change button 93 generates a mode signal to the lighting control unit 92 for changeover, and is disposed, for example, on a front panel of the light source apparatus 85 or the processing apparatus 12, or on the grip handle 17 of the endoscope apparatus 11. In a manner similar to the lighting control unit 42 of the first embodiment, the lighting control unit 92 controls the drivers 50-52 to turn on and off the color LEDs 43-45 or adjust their light amounts. The lighting control unit 92 also controls movement of the moving mechanism 91 according to the mode signal from the mode change button 93.

The moving mechanism 91 is constituted by a motor, and a rack and pinion gear (not shown) for changing rotational force of the motor into linear movement. The moving mechanism 91 slides the wavelength cut-off filter 48 between an operative position of the solid line in front of the blue semiconductor light source 35 and an inoperative position of the broken line beside the front of the blue semiconductor light source 35.

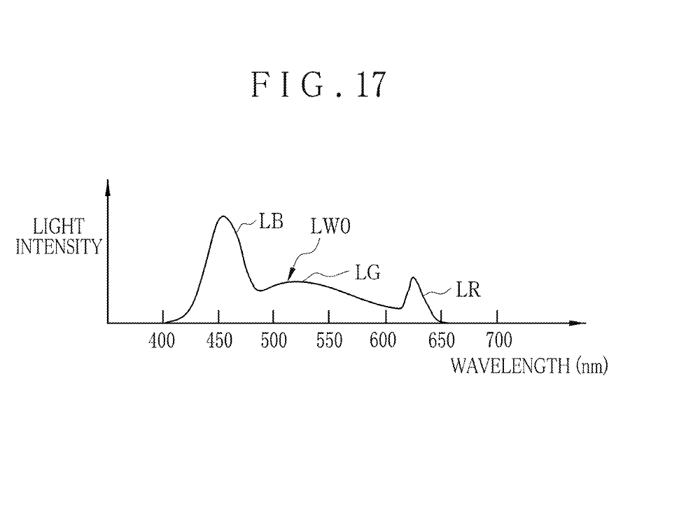

While the wavelength cut-off filter 48 is in the operative position to enable a cut-off function, the blue light LB is converted into the filtered blue light LB1c by cutting off a component of a wavelength equal to or longer than 455 nm or peak wavelength. An object of interest is illuminated by the illumination light LW1 as mixture of the filtered blue light LB1c, green light LG and red light LR. While the wavelength cut-off filter 48 is in the inoperative position to disable the cut-off function, the blue light LB becomes incident on the optical coupling device 41 without change. The object of interest is illuminated by the illumination light LW0 of FIG. 17 as mixture of the blue, green and red light LB, LG and LR.

The illumination light LW0 is constituted by combining the blue light LB to the green and red light LG and LR, and has a spectral distribution near to white light applied for imaging of the entirety of the object of interest according to the known imaging. The illumination light LW0 is different from the illumination light LW1 after filtering of the blue light LB for raising the contrast of surface blood vessels, and is suitable for imaging of the entirety of the object of interest. Also, a light amount of the illumination light LW0 is larger than that of the illumination light LW1 because of keeping the component of the blue light LB without the wavelength cut-off.

The use of the mode changer 90 enables or disables the cut-off function of the wavelength cut-off filter 48 by manual operation of a physician or user. It is possible to image the entirety of the object of interest with white light as is well-known (normal mode), and to image the surface blood vessels in enhanced manner (enhancement mode). For example, the normal mode is selected at an initial imaging step for imaging the entirety of the object of interest. Should a lesion be discovered in the object of interest, the enhancement mode can be selected. For imaging the entirety of the object of interest, the tip device 19 can be set distant from the object of interest to image the object of interest at a relatively far distance. The use of the illumination light LW0 with a larger light amount than the illumination light LW1 is advantageous.

The spectral distribution of the illumination light is different between the normal mode and the enhancement mode. It is preferable to set the condition of the signal processing in the digital signal processor 66, such as white balancing, for example, in consideration of equality in the color balance of an endoscopic image between the normal mode and the enhancement mode.

In the above embodiment, the lighting control unit 92 controls the moving mechanism 91. However, an additional controller can be provided and control the moving mechanism 91 besides the lighting control unit 92.

The moving mechanism for the wavelength cut-off filter 48 is not limited to the above structure. For example, a turret or disk of glass in a transparent form is prepared. The wavelength cut-off filter 48 is formed on a half area of the turret. A second half area of the turret remains transparent for passage of blue light LB. A motor rotates the turret to enable or disable the wavelength cut-off of the wavelength cut-off filter 48.

The wavelength cut-off filter 48 is not limited to that in the above embodiments without change in a transmission characteristic. For example, the wavelength cut-off filter 48 can be an etalon filter, a liquid crystal tunable filter and the like of which the transmission characteristic is variable. The etalon filter includes an actuator and two high reflectivity optical filters. Driving the actuator, such as a piezoelectric device, changes an interval between plate surfaces of the high reflectivity optical filters so as to control a wavelength range of transmitted light. The liquid crystal tunable filter includes two polarization filters, and a birefringent filter and nematic liquid crystal cells disposed between the polarization filters. A voltage applied to the liquid crystal cells is varied to control the wavelength range of transmitted light. The filters with the variable transmission characteristic such as the etalon filter and the liquid crystal tunable filter are advantageous in a low cost and small space because of absence of a moving mechanism for a wavelength cut-off filter (LCF). In relation to the use of the filters with the variable transmission characteristic such as the etalon filter and the liquid crystal tunable filter, a mode changer of the second embodiment is constituted by a driver and a controller, the driver driving the etalon filter or the liquid crystal tunable filter to change the wavelength range of transmitted light, the controller causing the driver to control the etalon filter or the liquid crystal tunable filter.

### Third Preferred Embodiment

In the above embodiments, the semiconductor light sources 35-37 are used. In another preferred embodiment, a violet semiconductor light source is added, for the purpose of imaging particular surface blood vessels nearer to the mucosa surface among the surface blood vessels.

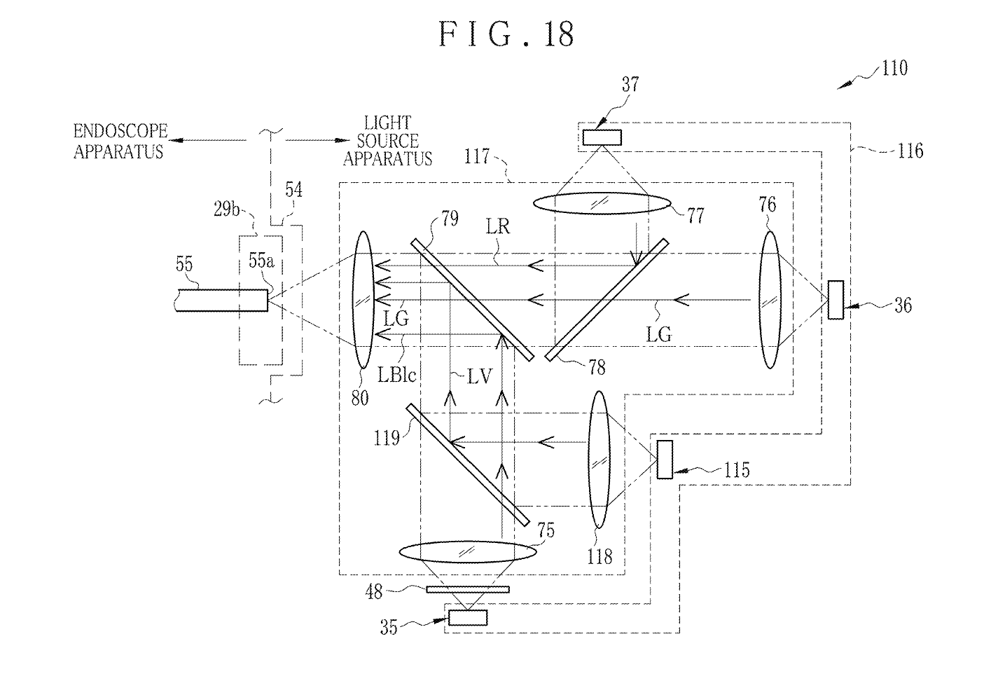

In FIG. 18, a light source apparatus 110 includes an illuminator 116 or light source unit, and an optical coupling device 117 or light coupling device in addition to the semiconductor light sources 35-37. A violet semiconductor light source 115 is incorporated in the illuminator 116. The optical coupling device 117 combines light paths from the semiconductor light sources 35-37 and the violet semiconductor light source 115 with one another. Except for portions of the illuminator 116 and the optical coupling device 117, the light source apparatus 13 of the first embodiment is repeated in the light source apparatus 110. Elements similar to those of the above embodiment are designated with identical reference numerals.

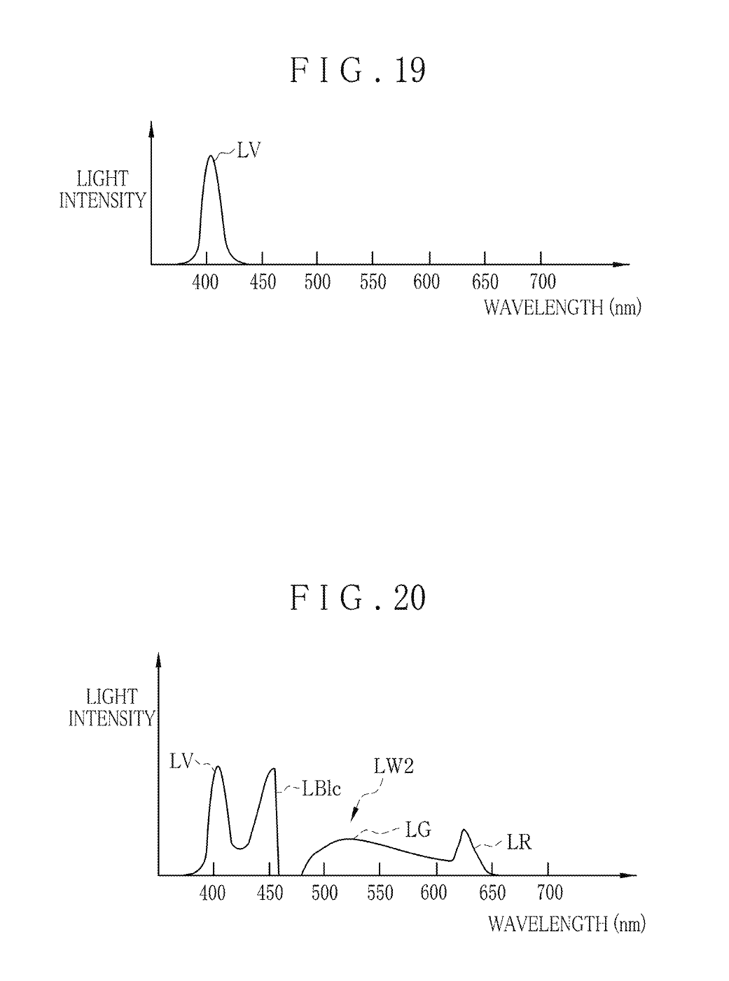

The violet semiconductor light source 115 includes a violet LED or violet light emitting device (not shown) for emitting light of a violet wavelength range. For the violet semiconductor light source 115, a structure of the blue semiconductor light source 35 in FIG. 4 is repeated. As illustrated in FIG. 19, the violet semiconductor light source 115 emits violet light LV of a wavelength of 395-415 nm in the violet color with a center wavelength of 405±10 nm and a peak wavelength of 405 nm.

The optical coupling device 117 includes a collimator lens 118 and a dichroic mirror 119 in addition to the structure of the optical coupling device 41. The collimator lens 118 collimates the violet light LV. The dichroic mirror 119 couples light paths of filtered blue light LB1c and violet light LV with one another. The optical coupling device 117 couples light paths from the filtered blue light LB1c, green light LG, red light LR and violet light LV with one another to constitute one downstream light path. In FIG. 20, spectral distribution of combined light (mixed light) of the components from the optical coupling device 117 is illustrated, the components including the filtered blue light LB1c, green light LG, red light LR and violet light LV. The combined light is used as illumination light LW2.

The blue and violet semiconductor light sources 35 and 115 are so arranged that their optical axes are perpendicular to one another. The dichroic mirror 119 is positioned at an intersection of those optical axes. The dichroic mirror 119 is inclined at 45 degrees with reference to the optical axes of the blue and violet semiconductor light sources 35 and 115.

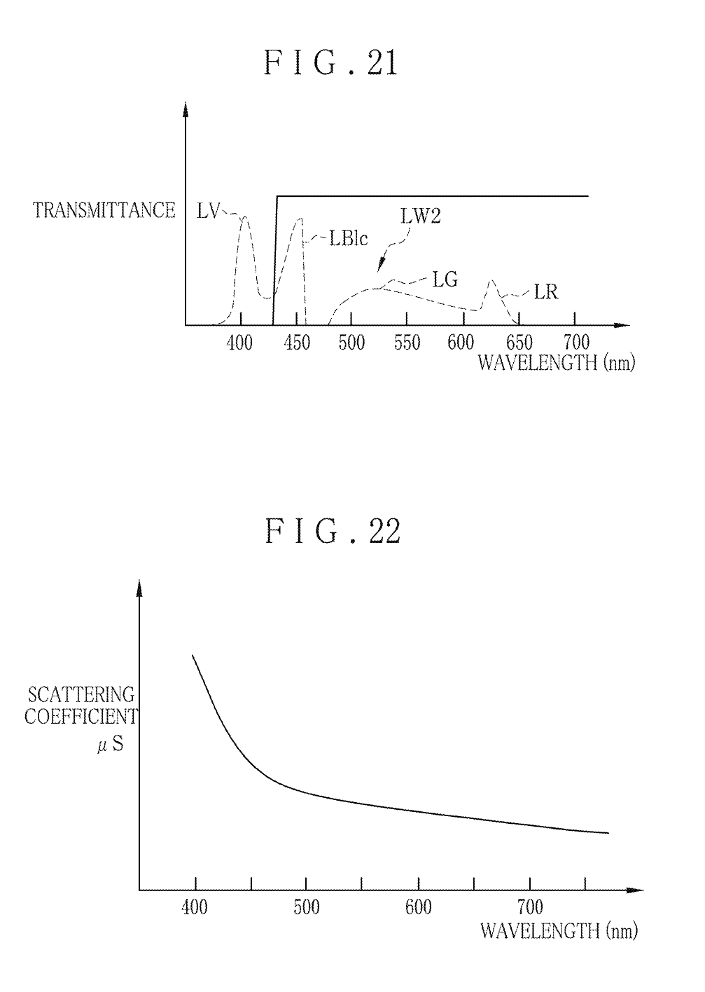

In FIG. 21, a dichroic filter of the dichroic mirror 119 characteristically reflects light of a wavelength shorter than approximately 430 nm in a violet color and transmits light of a wavelength equal to or longer than approximately 430 nm in blue, green and red colors. The dichroic mirror 119 transmits the filtered blue light LB1c passed through the collimator lens 75. Also, the dichroic mirror 119 reflects the violet light LV from the violet semiconductor light source 115 and the collimator lens 118. The dichroic mirror 119 combines light paths of the violet light LV and the filtered blue light LB1c with one another. In FIG. 15, the second dichroic mirror 79 characteristically reflects light of a wavelength shorter than approximately 470 nm in a blue color. The violet light LV reflected by the dichroic mirror 119 is reflected by the second dichroic mirror 79 to travel toward the condenser lens 80. Consequently, all of the light paths of those are coupled together.

In FIG. 24, reflectance of surface blood vessels decreases remarkably in a wavelength range under 450 nm (characteristic wavelength), and is the lowest at approximately 405 nm. Upon applying light of a low wavelength range and low reflectance to an object of interest, an endoscopic image of a difference of contrast between blood vessels and other tissue can be obtained, because of high absorption along blood vessels.

In FIG. 22, a characteristic of scattering light in body tissue is dependent upon the wavelength of light. A scattering coefficient μS increases according to shortness of the wavelength. The scattering influences to penetration depth of light into the body tissue. An amount of light reflected near to the mucosa of the body tissue is high according to the greatness of the scattering, to lower the amount of light reached to a deep layer or a medium depth layer of the body tissue. Accordingly, the penetration depth is lower according to the decrease of the wavelength, and higher according to the increase of the wavelength.

The violet light LV from the violet semiconductor light source 115 with the central wavelength of 405±10 nm has a relatively short wavelength and small penetration depth, and is absorbable in particular surface blood vessels nearer to the mucosa surface among the surface blood vessels, which are imaged in the above-described embodiments. Thus, the violet light LV is used as special light for enhancement of the particular surface blood vessels. The use of the violet light LV can obtain an image in which the particular surface blood vessels are expressed at a high contrast in addition to the surface blood vessels enhanced with the filtered blue light LB1c.

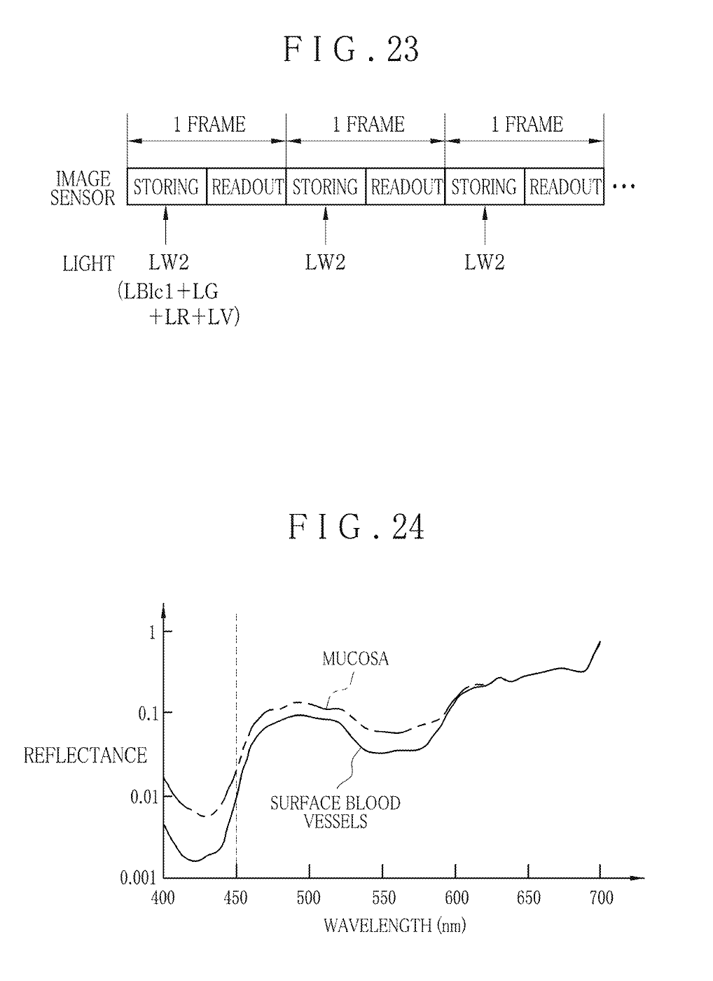

In FIG. 23, the violet semiconductor light source 115 is turned on in addition to the semiconductor light sources 35-37 in synchronism with the storing of the image sensor 56 for the purpose of image enhancement of the particular surface blood vessels. The violet light LV is added to the illumination light LW1 upon turning on of the semiconductor light sources 35-37 and 115, to apply illumination light LW2 of FIG. 20 to the object of interest as combined light (LW1+LV) or mixed light.

The illumination light LW2 after adding the violet light LV to the illumination light LW1 is separated by the micro color filter in the image sensor 56. Blue pixels receive the reflected light according to the filtered blue light LB1c and also the reflected light according to the violet light LV. In a manner similar to the first embodiment, green pixels receive the reflected light according to the green light LG. Red pixels receive the reflected light according to the red light LR. The image sensor 56 outputs the image signals B, G and R at the frame rate in response to the readout sequentially.

The image signal B is formed after using the components of reflected light according to the filtered blue light LB1c in the illumination light LW1 and also of reflected light according to the violet light LV. Thus, the particular surface blood vessels nearer to the mucosa surface can be expressed at a high contrast in addition to the surface blood vessels. It is medically known that there is a characteristic pattern of the particular surface blood vessels in body tissue of a cancer, malignant tumor or other lesions, because higher vessel density of the particular surface blood vessels is found than normal body tissue. According to the light source apparatus 110 of the embodiment, the particular surface blood vessels can be expressed distinctly with advantages for the diagnosis of a benign or malignant tumor.

In the first embodiment, values of drive currents for the color LEDs 43-45 are controlled to control the light amounts. However, it is likely that output light amounts of the color LEDs 43-45 may change due to various factors, such as heat of the LEDs, long time of use, and the like. In view of this, it is possible to use photo sensors for measurement of light amounts of the color light so as to monitor the value of the light amounts with reference to target light amount according to detection signals from the photo sensors.

The light source controller compares the light amount signal with a target light amount, and finely adjusts drive currents for the semiconductor light sources 35-37 according to the exposure control so as to set the light amount equal to the target light amount by considering the result of the comparison. It is possible to control the light amount always to reach the target light amount by the fine adjustment of the drive currents according to monitoring of the light amounts with the photo sensor for measurement. This is effective in obtaining illumination light of stabilized spectral distribution as target.

In the above embodiments, the light sources are LEDs as semiconductor light sources. However, a green light source may include a blue LED and green phosphor, the blue LED emitting blue light as an excitation source in colors from violet to blue, the green phosphor being excited by the blue light and emitting green light. In addition to this or in place of this, a red light source may include a blue LED and red phosphor, the blue LED emitting blue light as an excitation source in colors from violet to blue, the red phosphor being excited by the blue light and emitting red light. For such examples, a green LED can be used for excitation in place of the blue LED, emitting green light as an excitation source. See FIG. 4 of the first embodiment. Phosphor is filled in the cavity in the cavity mold 35b instead of the resin encapsulant 35c, to constitute a semiconductor light source having the phosphor.

The mounting type of the LED in FIG. 4 is only an example and can be modified. For example, a micro lens can be disposed at an exit surface of the resin encapsulant 35c for adjusting the divergence angle. Also, a case or housing can be formed in a bullet shape with a micro lens, and can contain the LED in a manner different from the surface mounting type. Note that phosphor in fluorescent light sources (semiconductor) can be separately disposed from excitation sources (LEDs) in the use of the fluorescent light sources as green and red semiconductor light sources, in a manner different from the fluorescent light sources above in which the phosphor is mounted together with the excitation sources. To this end, a lens, optical fiber or other optics for guiding light is added between the excitation sources and the phosphor, so that excitation light from the excitation sources is guided to the phosphor.

In the phosphor type of light sources, laser diodes (LD) can be used instead of the LEDs. Also, organic electro luminescence devices (EL) may be used. For light sources other than the phosphor type, it is possible to use laser diodes (LD) or organic electro luminescence devices (EL) in light sources except for the blue semiconductor light source 35.

In the above embodiments, the wavelength cut-off filter 48 cuts off 100% of the long wavelength component of the blue light LB equal to or longer than the reference wavelength. However, the present invention is not limited to the example. A long wavelength component can be cut off at least partially in the range equal to or longer than the peak wavelength in the blue light LB. Also, an optical filter in place of the wavelength cut-off filter 48 can be such a structure as to reduce a light amount of a long wavelength component of the blue light LB. For example, an optical filter may cut off 50% of the long wavelength component of the blue light LB. However, cutting off of 100% of the long wavelength component of the blue light LB is the most preferable in view of effectively raising the contrast of surface blood vessels.

Various modifications are possible in the features of the optical coupling device. In the above embodiments, the dichroic mirrors with the dichroic filters are used. Instead, a dichroic prism in which a dichroic filter is formed on a prism can be used. Furthermore, in place of the optics with the dichroic filters, a coupling light guide device of a branch form can be used for combining light paths, including a plurality of entrance ends downstream of the semiconductor light sources, and one exit end upstream of the light guide device of the endoscope. The coupling light guide device is a fiber bundle including plural optical fibers of a bundle. At the entrance ends, the fibers are grouped in plural groups for branches. The semiconductor light sources are suitably disposed in connection with the entrance ends.

Furthermore, the green light LG may be used mainly in illumination light for application to an object of interest. The illumination light can be constituted by mixing the filtered blue light LB1c with the green light LG, mixing the violet light LV with the green light LG, and the like.

In the above embodiments, the micro color filters of B, G and R are used with the image sensor 56 as a color imaging device. However, an image sensor in the invention may include a monochromatic image sensor. Three color light sources can be combined and driven for emitting B, G and R light sequentially. Image signals of B, G and R can be acquired, in a form of an endoscope system of a frame sequential method of imaging.

Also, two or more of the features of the embodiments can be combined with one another.

In the above embodiment, the processing apparatus 12 is separate from the light source apparatus 13. However, a single apparatus in an endoscope system of the invention may include components of the light source apparatus 13 and the processing apparatus 12. A light source apparatus of the invention can be used with a fiberscope for guiding reflected light from an object of interest with an image guide device, an ultrasonic endoscope including an ultrasonic transducer in a tip device, and other endoscope apparatus.

Also, a medical apparatus for use with a light source apparatus of the invention can be a light delivery catheter and the like, in place of the endoscope apparatus. It is possible in the endoscope system of the invention to utilize various known techniques of a light delivery system and diagnosis support system for medical use.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A light source apparatus for outputting illumination light for an endoscope apparatus, comprising:
a blue semiconductor light source for emitting blue light with a peak wavelength equal to or longer than 450 nm;
a wavelength cut-off filter, disposed downstream of said blue semiconductor light source, for cutting off a component in said blue light having a wavelength equal to or longer than said peak wavelength.

2. A light source apparatus as defined in claim 1, wherein said wavelength cut-off filter cuts off a component defined at a wavelength equal to or longer than said peak wavelength.

3. A light source apparatus as defined in claim 1, wherein said wavelength cut-off filter cuts off a component with a wavelength on a long wavelength side of a wavelength range of a half width of said blue light.

4. A light source apparatus as defined in claim 1, further comprising a mode changer for changing over an enhancement mode and a normal mode, said enhancement mode being set for enhancing and imaging a surface blood vessel of said object by use of said wavelength cut-off filter, said normal mode being set for imaging said object by disabling a function of said wavelength cut-off filter.

5. A light source apparatus as defined in claim 4, wherein said mode changer includes:
a moving mechanism for moving said wavelength cut-off filter between an operative position inside a light path from said blue semiconductor light source and an inoperative position outside said light path;
a controller for driving said moving mechanism according to selection of said enhancement mode and said normal mode, for setting said wavelength cut-off filter in said operative position in case of selecting said enhancement mode, and for setting said wavelength cut-off filter in said inoperative position in case of selecting said normal mode.

6. A light source apparatus as defined in claim 1, further comprising:
a green semiconductor light source for emitting green light of a wavelength range of green;
a red semiconductor light source for emitting red light of a wavelength range of red;
an optical coupling device for combining said blue, green and red light from said wavelength cut-off filter, said green semiconductor light source and said red semiconductor light source with one another, to output said illumination light.

7. A light source apparatus as defined in claim 6, further comprising a violet semiconductor light source for emitting violet light of a wavelength range of violet, to enhance a surface blood vessel of said object at least partially.

8. A light source apparatus as defined in claim 6, wherein said optical coupling device includes:
a first dichroic mirror for combining light of a first color with light of a second color, said first and second colors being selected from said blue, green and red;
a second dichroic mirror for combining combined light from said first dichroic mirror with a third color selected from said blue, green and red, to output said illumination light.

9. A light source apparatus as defined in claim 1, wherein said blue semiconductor light source has a blue light-emitting diode.

10. An endoscope system having an endoscope apparatus and a light source apparatus for outputting illumination light for said endoscope apparatus, comprising:
said endoscope apparatus including:
a light guide device for transmitting said illumination light in a distal direction;
a lighting window area for applying said illumination light from said light guide device to an object in a body cavity;
said light source apparatus including:
a blue semiconductor light source for emitting blue light with a peak wavelength equal to or longer than 450 nm;
a wavelength cut-off filter, disposed downstream of said blue semiconductor light source, for cutting off a component in said blue light having a wavelength equal to or longer than said peak wavelength.

11. An endoscope system as defined in claim 10, wherein said wavelength cut-off filter cuts off a component defined at a wavelength equal to or longer than said peak wavelength.

12. An endoscope system as defined in claim 10, wherein said wavelength cut-off filter cuts off a component with a wavelength on a long wavelength side of a wavelength range of a half width of said blue light.

13. An endoscope system as defined in claim 10, further comprising a mode changer for changing over an enhancement mode and a normal mode, said enhancement mode being set for enhancing and imaging a surface blood vessel of said object by use of said wavelength cut-off filter, said normal mode being set for imaging said object by disabling a function of said wavelength cut-off filter.

14. An endoscope system as defined in claim 13, wherein said mode changer includes:
a moving mechanism for moving said wavelength cut-off filter between an operative position inside a light path from said blue semiconductor light source and an inoperative position outside said light path;
a controller for driving said moving mechanism according to selection of said enhancement mode and said normal mode, for setting said wavelength cut-off filter in said operative position in case of selecting said enhancement mode, and for setting said wavelength cut-off filter in said inoperative position in case of selecting said normal mode.
